(19) United States
(12) Patent Application Publication
(10) Pub. No.: US 2024/0423480 A1
(43) Pub. Date: Dec. 26, 2024

(54) MINIATURIZED TRANSPARENT PHOTOACOUSTIC SENSOR

(71) Applicant: QUALCOMM Incorporated, San Diego, CA (US)

(72) Inventors: Sumit AGRAWAL, Sunnyvale, CA (US); Jessica Liu STROHMANN, Cupertino, CA (US); Hrishikesh Vijaykumar PANCHAWAGH, Cupertino, CA (US); Kostadin Dimitrov DJORDJEV, Los Gatos, CA (US)

(21) Appl. No.: 18/340,821

(22) Filed: Jun. 23, 2023

(51) Int. Cl. A61B 5/00; A61B 5/02

(52) U.S. Cl. CPC A61B 5/0095; A61B 5/02007; A61B 2562/0204; A61B 2562/04; A61B 2562/182

(57) ABSTRACT

An apparatus and associated method of manufacture may include a receiver system that includes an array of receiver elements, in addition to receiver system circuitry. A least a portion of the array of receiver elements is transparent. The transparency allows light to pass through so that blood vessel behind may be visible or otherwise perceived. In this manner, the receiver system may detect (i.e., through the transparent portion of the receiver elements) acoustic waves corresponding to the blood vessel's photoacoustic response to light emitted by a light source system that includes a light-emitting component.

### TECHNICAL FIELD

This disclosure relates generally to photoacoustic devices and systems. log files are passed on as inputs to the hardware word-to-vector module.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive, and ambulatory monitoring. Some such devices are, or include photoacoustic devices. Although some previously-deployed photoacoustic devices and systems can provide acceptable results, improved photoacoustic devices and systems would be desirable.

### SUMMARY

The systems, methods, and devices of this disclosure each have several aspects, no single one of which is solely responsible for the desirable attributes disclosed herein.

One innovative aspect of the subject matter described in this disclosure can be implemented in an apparatus. The apparatus may include a receiver system that includes an array of receiver elements, in addition to receiver system circuitry. A least a portion of the array of receiver elements is transparent. The transparency allows light to pass through so that blood vessel behind may be visible or otherwise perceived. In this manner, the receiver system may detect (i.e., through the transparent portion of the receiver elements) acoustic waves corresponding to the blood vessel's photoacoustic response to light emitted by a light source system that includes a light-emitting component.

According to some implementations, a platen having a transparent platen portion may be included, wherein the platen is positioned in between the receiver system and where the blood vessel is presented. The thickness of the platen may range from 200 um to 400 um.

In some implementations, the array of receiver elements may include a transparent electrode layer. In one aspect, the receiver elements further comprise a piezoelectric layer having a first side on which the transparent electrode layer is included, and further comprising a patterned electrode layer included on a second opposite side of the piezoelectric layer. According to some aspects, a transparent matching layer may be positioned in between the receiver elements and the blood vessel.

In some implementations, a first transparent backing layer may be positioned in between the array of receiver elements and the light source system. The first transparent backing layer may comprise at least one of glass or epoxy. A second transparent backing layer may be proximate the first transparent backing layer. The first and second transparent backing layers may have different material compositions. A first material composition of the second transparent backing layer may be selected to affect at least one of an acoustic impedance or an attenuation based on a second material composition of the first transparent backing layer.

According to some aspects, a transparent substrate with an electromagnetic interference (EMI) shield may be positioned in between the array of receiver elements and the light source system. The apparatus may include a light guide positioned in between the array of receiver elements and the light source system. A lens may be positioned in between the array of receiver elements and the light source system. In another implementation, a lens may be positioned in between the array of receiver elements and where the blood vessel is presented. The receiver system of an implementation may include at least one of: lithium niobate, lead magnesium niobate-lead titanate (PMN-PT), polyvinylidene fluoride tetrafluoroethylene (PVDF), or a copolymer film with an indium tin oxide coating.

Other innovative aspects of the subject matter described in this disclosure can be implemented in an apparatus comprising: a means for emitting a light a means for presenting an array of receiver elements, wherein at least a portion of the array of receiver elements is transparent, and a means for detecting acoustic waves corresponding to a photoacoustic response of a blood vessel to the emitted light through the portion of the array of receiver elements that is transparent.

Other innovative aspects of the subject matter described in this disclosure can be implemented in a method. The method may involve positioning a light source system including a light-emitting component, and positioning a receiver system in proximity of the light source, wherein the light source system includes an array of receiver elements and receiver system circuitry, wherein at least a portion of the array of receiver elements is transparent, the receiver system being configured to detect acoustic waves corresponding to a photoacoustic response of a blood vessel to light emitted by the light source system through the transparent portion of the array of receiver elements.

According to another particular aspect, the method may include positioning a platen in between the receiver system and where the blood vessel is presented, wherein the platen has a transparent platen portion. The method may further include manufacturing the platen to have a thickness ranging from 200 um to 400 um.

According to an implementation, the method may include positioning the array of receiver elements with a transparent electrode layer. In another aspect, the method involves including within the receiver elements a piezoelectric layer having a first side on which the transparent electrode layer is positioned and a second patterned electrode layer positioned on a second opposite side of the piezoelectric layer. A transparent matching layer may be positioned in between the receiver elements and the blood vessel.

In another aspect, the method may include positioning a first transparent backing layer in between the array of receiver elements and the light source system. Another aspect may include manufacturing the first transparent backing layer from glass and epoxy. The method may further comprise positioning a second transparent backing layer proximate the first transparent backing layer.

According to a particular aspect, the method may include manufacturing the first and second transparent backing layers to have different material compositions. The method may include selecting a first material composition of the second transparent backing layer to affect at least one of an acoustic impedance or an attenuation based on a second material composition of the first transparent backing layer. Another aspect may include positioning a transparent substrate with an EMI shield in between the array of receiver elements and the light source system. The method may further include positioning a light guide in between the array of receiver elements and the light source system.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods.

Details of one or more implementations of the subject matter described in this disclosure are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1 is a block diagram with illustrative components of an apparatus according to some disclosed implementations.

FIG. 2 shows some elements of a PAPG-capable sensor device having a receiver system stack that includes multiple transparent backing layers, among other components.

FIG. 3 shows another implementation of elements of a PAPG-capable sensor device having a receiver system stack that unlike with the apparatus of FIG. 2, the apparatus of FIG. 3 includes no transparent backing layers.

FIG. 4 is a block diagram of a side view of a photoacoustic sensor that includes, among other transparent components, a light guide, a transparent backing layer, and transparent patterned electrodes.

FIG. 5 is a block diagram of a side view of another photoacoustic sensor that differs from the apparatus of FIG. 4 by including an additional patterned electrode layer.

FIG. 6 shows some elements of a PAPG-capable sensor device having a receiver system stack that includes an optical focus lens positioned above a light source system.

FIG. 7 shows some elements of a PAPG-capable sensor device having a receiver system stack that includes an optical focus lens positioned above the PAPG stack.

FIG. 8 shows examples of a device that may be used in a system for estimating blood pressure.

FIG. 9 shows examples of heart rate waveform (HRW) features that may be extracted according to some implementations of the systems and method described herein.

FIG. 10 shows examples of devices that may be used in a system for estimating blood pressure based, at least in part, on pulse transit time (PTT).

FIG. 11 shows a cross-sectional side view of a diagrammatic representation of a portion of an artery through which a pulse is propagating.

FIG. 12A shows an example ambulatory monitoring device designed to be worn around a wrist according to some implementations.

FIG. 12B shows an example ambulatory monitoring device designed to be worn on a finger according to some implementations.

FIG. 12C shows an example ambulatory monitoring device designed to reside on an earbud according to some implementations.

FIG. 13 is a flow diagram that shows examples of some disclosed operations

Like reference numbers and designations in the various drawings indicate like elements

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Non-invasive health monitoring devices, such as photoacoustic plethysmography (PAPG)-capable devices, have various potential advantages over more invasive health monitoring devices such as cuff-based or catheter-based blood pressure measurement devices. However, it has proven to be difficult to design satisfactory PAPG-capable devices. For example, a thick platen is typically positioned above the receiver in conventional configurations. Typical platens can be constructed of 15 mm thick tungsten. This thick, opaque platen composition can result in a bulky PAPG sensor module. The dimensions of the PAPG may present a challenge when used as a wearable device.

An implementation described herein couples a vertical-cavity surface-emitting laser (VCSEL) source with a transparent acoustic receiver in PAPG sensors. An apparatus and associated method of manufacture may include a receiver system that includes an array of receiver elements, in addition to receiver system circuitry. A least a portion of the array of receiver elements is transparent. The transparency allows light to pass through so that blood vessel behind may be visible or otherwise perceived. In this manner, the receiver system may detect (i.e., through the transparent portion of the receiver elements) acoustic waves corresponding to the blood vessel's photoacoustic response to light emitted by a light source system that includes a light-emitting component.

Particular implementations of the subject matter described in this disclosure can be implemented to realize one or more of the following potential advantages. Various disclosed configurations enable light delivery into tissue in a manner that results in higher sensitivity towards the estimation of arterial blood pressure. An implementation may enhance the sensitivity of the device to received photoacoustic waves, such as arterial photoacoustic waves. An implementation may allow co-alignment of optical and acoustic paths. According to some implementations, acoustic impedance matching layers may mitigate unwanted reflections of acoustic waves, thereby mitigating another type of noise. The implementation may allow light coming out of the source to directly hit the tissue region and a photoacoustic signal is generated from the absorbing target tissue (e.g., a radial artery). The generated photoacoustic wave travels isotopically and is received by the transparent ultrasonic receiver as a strong signal.

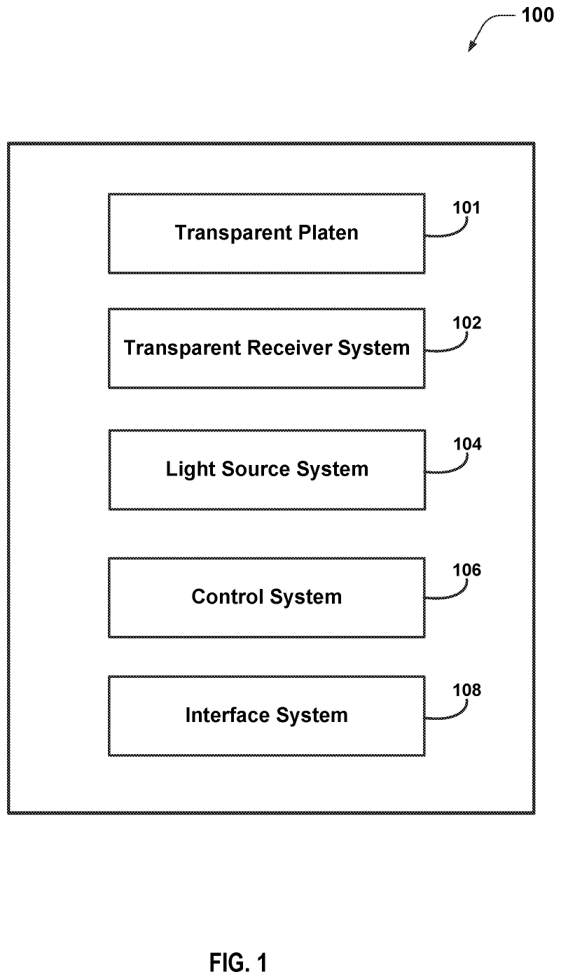

FIG. 1 is a block diagram that shows example components of an apparatus according to some disclosed implementations. In this example, the apparatus 100 includes a transparent platen 101, a transparent receiver system 102, and a light source system 104. Some implementations of the apparatus 100 may include a control system 106, an interface system 108, or both.

The transparent platen 101 may be made of any suitable material, such as glass, acrylic, polycarbonate, etc. According to some examples, the transparent platen 101 (or another portion of the apparatus) may include one or more anti-reflective layers. In some examples, one or more anti-reflective layers may reside on, or proximate, one or more outer surfaces of the transparent platen 101.

In some examples, at least a portion of the outer surface of the transparent platen 101 may have an acoustic impedance that is configured to approximate an acoustic impedance of human skin. The portion of the outer surface of the transparent platen 101 may, for example, be a portion that is configured to receive a target object, such as a human digit. (As used herein, the terms “finger” and “digit” may be used interchangeably, such that a thumb is one example of a finger.) A typical range of acoustic impedances for human skin is 1.53-1.680 MRayls. In some examples, at least an outer surface of the transparent platen 101 may have an acoustic impedance that is in the range of 1.4-1.8 MRayls, or in the range of 1.5-1.7 MRayls. Alternatively, or additionally, in some examples at least an outer surface of the transparent platen 101 may be configured to conform to a surface of human skin. In some such examples, at least an outer surface of the transparent platen 101 may have material properties like those of putty or chewing gum.

In some examples, at least a portion of the transparent platen 101 may have an acoustic impedance that is configured to approximate an acoustic impedance of one or more receiver elements of the receiver system 102. According to some examples, a layer residing between the transparent platen 101 and one or more receiver elements may have an acoustic impedance that is configured to approximate an acoustic impedance of the one or more receiver elements. Alternatively, or additionally, in some examples a layer residing between the transparent platen 101 and one or more receiver elements may have an acoustic impedance that is in an acoustic impedance range between an acoustic impedance of the platen and an acoustic impedance of the one or more receiver elements.

According to some examples, the receiver system 102 may include an array of receiver elements, in addition to receiver system circuitry. A least a portion of the array of receiver elements is transparent. The transparency allows light to pass through so that blood vessel behind may be visible or otherwise perceived. In this manner, the receiver system may detect (i.e., through the transparent portion of the receiver elements) acoustic waves corresponding to the blood vessel's photoacoustic response to light emitted by a light source system that includes a light-emitting component.

In this implementation, the receiver system 102 is, or includes an ultrasonic receiver system. In some examples, the receiver system 102 may be configured to detect acoustic waves corresponding to a photoacoustic response of the target object to light emitted by the light source system. In some examples, the receiver system 102 may include a piezoelectric receiver layer, such as a layer of polyvinylidene difluoride (PVDF) polymer, polyvinylidene fluoride-trifluoroethylene (PVDF-TrFE) copolymer, a piezoelectric composite, etc. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 102 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 102 may be, or may include an ultrasonic receiver array. In some examples, the apparatus 100 may include one or more separate ultrasonic transmitter elements. In some such examples, the ultrasonic transmitter(s) may include an ultrasonic plane-wave generator. In some examples, the receiver system 102 may include an optical receiver system.

According to some examples, the light source system 104 may include a light-emitting component and light source system circuitry. In some examples, the light source system 104 may be configured to emit light through a transparent portions(s) of the transparent receiver system 102 towards a target object in contact with the first area of the transparent platen 101.

The light source system 104 may, in some examples, include one or more light-emitting diodes. In some implementations, the light source system 104 may include one or more laser diodes. According to some implementations, the light source system 104 may include one or more vertical-cavity surface-emitting lasers (VCSELs). In some implementations, the light source system 104 may include one or more edge-emitting lasers. In some implementations, the light source system may include one or more neodymium-doped yttrium aluminum garnet (Nd:YAG) lasers.

The light source system 104 may, in some examples, be configured to transmit light in one or more wavelength ranges. In some examples, the light source system 104 may configured for transmitting light in a wavelength range of 500 to 600 nanometers. According to some examples, the light source system 104 may configured for transmitting light in a wavelength range of 800 to 950 nanometers.

The light source system 104 may include various types of drive circuitry, depending on the particular implementation. In some disclosed implementations, the light source system 104 may include at least one multi-junction laser diode, which may produce less noise than single-junction laser diodes. In some examples, the light source system 104 may include a drive circuit (also referred to herein as drive circuitry) configured to cause the light source system to emit pulses of light at pulse widths in a range from 3 nanoseconds to 1000 nanoseconds. According to some examples, the light source system 104 may include a drive circuit configured to cause the light source system to emit pulses of light at pulse repetition frequencies in a range from 1 kilohertz to 100 kilohertz.

In some implementations, the light source system 104 may be configured for emitting various wavelengths of light, which may be selectable to trigger acoustic wave emissions primarily from a particular type of material. For example, because the hemoglobin in blood absorbs near-infrared light very strongly, in some implementations the light source system 104 may be configured for emitting one or more wavelengths of light in the near-infrared range, in order to trigger acoustic wave emissions from hemoglobin. However, in some examples the control system 106 may control the wavelength(s) of light emitted by the light source system 104 to preferentially induce acoustic waves in blood vessels, other soft tissue, and/or bones. For example, an infrared (IR) light-emitting diode LED may be selected, and a short pulse of IR light emitted to illuminate a portion of a target object and generate acoustic wave emissions that are then detected by the receiver system 102. In another example, an IR LED and a red LED or other color such as green, blue, white, or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 104 may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system 104 may be configured for emitting a plurality of light pulses at a pulse repetition frequency between about 1 MHz and about 100 MHz. Alternatively, or additionally, in some implementations the light source system 104 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 104 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 104. In some implementations, the light source system may contain light sources such as red, green, and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

In some implementations, the apparatus (for example, the receiver system 102, the light source system 104, or both) may include one or more sound-absorbing layers, acoustic isolation material, light-absorbing material, light-reflecting material, or combinations thereof. In some examples, acoustic isolation material may reside between the light source system 104 and at least a portion of the receiver system 102. In some examples, the apparatus (for example, the receiver system 102, the light source system 104, or both) may include one or more electromagnetically shielded transmission wires. In some such examples, the one or more electromagnetically shielded transmission wires may be configured to reduce electromagnetic interference from the light source system 104 that is received by the receiver system 102.

The control system 106 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. The control system 106 may be configured for receiving and processing data from the receiver system 102, e.g., as described below. If the apparatus 100 includes an ultrasonic transmitter, the control system 106 may be configured for controlling the ultrasonic transmitter. In some implementations, functionality of the control system 106 may be partitioned between one or more controllers or processors, such as a dedicated sensor controller and an applications processor of a mobile device.

In some examples, the control system 106 may be configured to control the light source system 104. For example, the control system 106 may be configured to control one or more light-emitting portions of the light source system 104 to emit laser pulses. The laser pulses may, in some examples, be in a wavelength range of 600 nm to 1000 nm. The laser pulses may, in some examples, have pulse widths in a range from 3 nanoseconds to 1000 nanoseconds. In some examples, the control system 106 may be configured to receive signals from the ultrasonic receiver system 102 corresponding to the ultrasonic waves generated by the target object responsive to the light from the light source system 104. In some examples, the control system 106 may be configured to estimate one or more cardiac features based, at least in part, on the signals. According to some examples, the cardiac features may be, or may include blood pressure.

Some implementations of the apparatus 100 may include the interface system 108. In some examples, the interface system 108 may include a wireless interface system. In some implementations, the interface system 108 may include a user interface system, one or more network interfaces, one or more interfaces between the control system 106 and a memory system and/or one or more interfaces between the control system 106 and one or more external device interfaces (e.g., ports or applications processors), or combinations thereof. According to some examples in which the interface system 108 is present and includes a user interface system, the user interface system may include a microphone system, a loudspeaker system, a haptic feedback system, a voice command system, one or more displays, or combinations thereof. According to some examples, the interface system 108 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 108 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. In some examples, the interface system 108 may include an optical sensor system, one or more cameras, or a combination thereof.

The apparatus 100 may be used in a variety of different contexts, many examples of which are disclosed herein. For example, in some implementations a mobile device may include the apparatus 100. In some such examples, the mobile device may be a smart phone. In some implementations, a wearable device may include the apparatus 100. The wearable device may, for example, be a bracelet, an armband, a wristband, a watch, a ring, a headband, or a patch.

Several implementation are described herein that illustrate various stack configurations of transparent receiver systems. Each receiver system described herein includes an array of receiver elements, in addition to receiver system circuitry. A least a portion of the array of receiver elements is transparent. “Transparent,” as used herein, may include a material that allows light to pass through so that blood vessel behind may be visible or otherwise perceived. In this manner, the receiver system may detect (i.e., through the transparent portion of the receiver elements) acoustic waves corresponding to the blood vessel's photoacoustic response to light emitted by a light source system that includes a light-emitting component. Not all of the components of a PAPG sensors are shown in each illustration, as the drawings are intended to include those components useful in describing the context and functionality of the features. Further, the respective thicknesses and other dimensions of the different stacked layers described herein are not to scale.

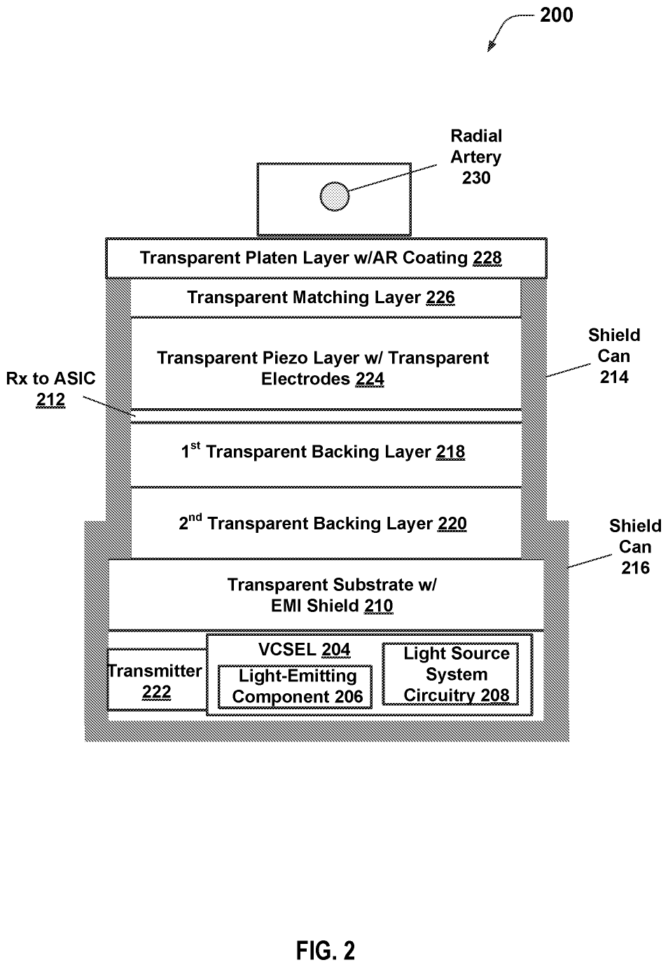

FIG. 2 shows some elements of a PAPG-capable sensor device having a receiver system stack that includes multiple transparent backing layers, among other components. The implementation may allow co-alignment of optical and acoustic paths. As with other disclosed examples, the types, numbers, and arrangements of elements shown in FIG. 2 are merely examples for illustrative purposes. Other examples may include different numbers of elements, types of elements, arrangements of elements, or combinations thereof.

In this example, the apparatus 100 includes a light source system that comprising a VCSEL 204 having a light-emitting component 206 and light source system circuitry 208. The VCSEL 204 may generate pulsed laser transmissions. According to this example, the apparatus 200 additionally includes a transparent substrate with an EMI shield 210. The EMI shield 210 may limit electromagnetic interference from impacting a layer 212 that includes a receiver 212 that is in communication with an application-specific integrated circuit (ASIC). The stack may be contained within shield cans 214, 216.

The apparatus 200 may additionally include first and second transparent backing layers 218, 220. The first and second transparent backing layers 218, 220 may comprise glass (e.g., beads) and transparent epoxy in one illustrative configuration. The first and second transparent backing layers 218, 220 may have different material compositions (e.g., compositions of transparent composites). For example, one transparent backing layer may have different sized or shaped glass particles. For instance, a configuration may include glass triangular structures. Different weight percentage of glass and epoxy may impact the acoustic impedance and attenuation. Both acoustic impedance and attenuation may be tuned using the transparent backing layers 218, 220 to affect overall optical transmission.

A transparent piezo layer with transparent electrodes 224 may be positioned above the receiver component layer 212. The receiver component layer 212 and transparent electrodes 224 (as with any other components included in the stack) may be constructed from materials that include: lithium niobate, lead magnesium niobate-lead titanate (PMN-PT), polyvinylidene fluoride tetrafluoroethylene (PVDF), and/or a copolymer film with an indium tin oxide coating. The receiver may detect acoustic waves corresponding to a photoacoustic response of a radial artery to laser light emitted by the VCSEL 204 through the transparent portions of the PAPG stack.

A transparent matching layer 226 may mitigate unwanted reflections of acoustic waves, thereby mitigating another type of noise. A transparent platen layer 228 may include an antireflective (AR) coating. The transparent platen layer 228 may be relatively thin (e.g., 200 um-400 um) compared to conventional tungsten platens. A radial artery 230 may be present proximate the transparent platen layer 228 to allow the system to determine blood pressure readings, among other data, from the detected acoustic waves.

The implementation may allow light coming out of the VCSEL 204 to directly hit the tissue region and a photoacoustic signal is generated from the absorbing target tissue (e.g., radial artery 230). The generated photoacoustic wave travels isotopically and is received by the transparent ultrasonic receiver (Rx) layer 212.

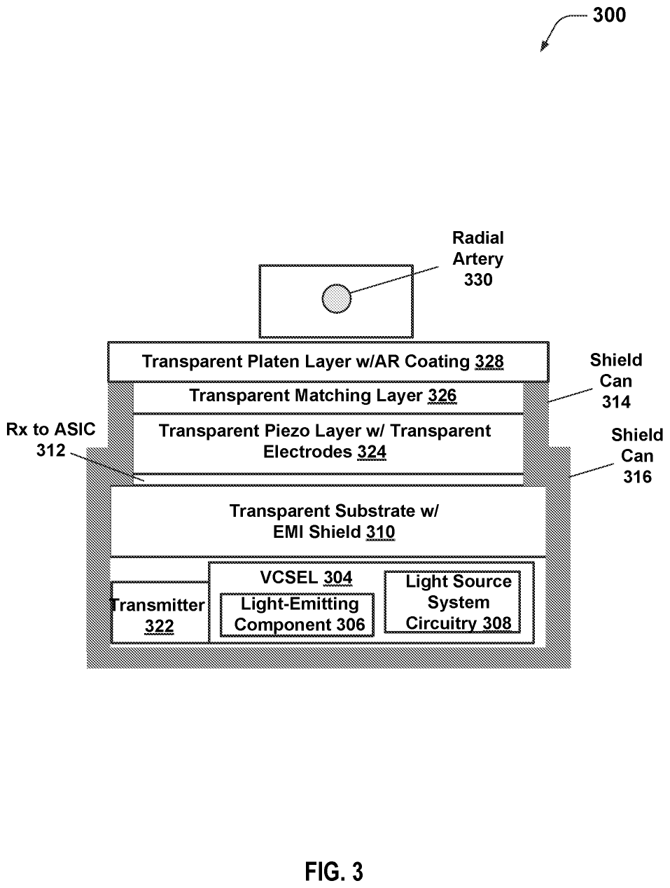

FIG. 3 shows another implementation of elements of a PAPG-capable sensor device having a receiver system stack. Unlike the apparatus 200 of FIG. 200, the apparatus 300 of FIG. 3 includes no transparent backing layers. The apparatus may use a controller, such as the control system 106 of FIG. 1, to apply an algorithm to modify signals received (e.g., to execute instructions to adjust the acoustic impedance and attenuation) in the absence of a backing layer. As with other disclosed examples, the types, numbers, and arrangements of elements shown in FIG. 3 are merely examples for illustrative purposes. Other examples may include different numbers of elements, types of elements, arrangements of elements, or combinations thereof.

In this example, the apparatus 300 includes a light source system that comprising a VCSEL 304 having a light-emitting component 306 and light source system circuitry 308. According to this example, the apparatus 300 additionally includes a transparent substrate with an EMI shield 310. The EMI shield 310 may limit electromagnetic interference from impacting a receiver layer 312 that is in communication with an application-specific integrated circuit (ASIC). The stack may be contained within shield cans 314, 316.

A transparent piezo layer with transparent electrodes 324 may be positioned above the receiver component layer 312. The receiver component layer 312 and transparent electrodes 324 (as with any other components included in the stack) may be constructed from materials that include: lithium niobate, lead magnesium niobate-lead titanate (PMN-PT), polyvinylidene fluoride tetrafluoroethylene (PVDF), and/or a copolymer film with an indium tin oxide coating. The receiver may detect acoustic waves corresponding to a photoacoustic response of a radial artery to laser light emitted by the VCSEL 304 through the transparent portions of the PAPG stack.

A transparent matching layer 326 may mitigate unwanted reflections of acoustic waves, thereby mitigating another type of noise. A transparent platen layer 328 may include an antireflective (AR) coating. A radial artery 330 may be present proximate the transparent platen layer 328 to allow the system to determine blood pressure readings, among other data, from the detected acoustic waves.

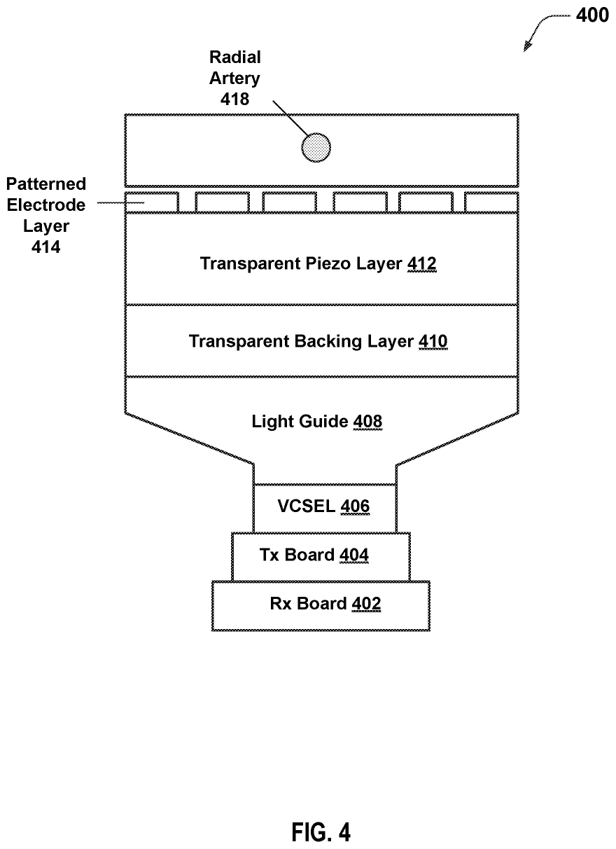

FIG. 4 is a block diagram of a side view of a photoacoustic sensor that includes, among other transparent components, a light guide, a transparent backing layer, and transparent patterned electrodes. The implementation may allow co-alignment of optical and acoustic paths. As with other disclosed examples, the types, numbers, and arrangements of elements shown in FIG. 4 are merely examples for illustrative purposes. Other examples may include different numbers of elements, types of elements, arrangements of elements, or combinations thereof.

Turning more particularly to the drawing, the apparatus 400 includes a receiver board 402 and a transmitter board 404. A VCSEL 406, which may generate pulsed laser transmissions, may be coupled to a light guide 408. The light guide 408 may include any suitable material, or combination of materials, for causing at least some of the light emitted by the VCSEL 406 to propagate within the light guide 408, for example due to total internal reflection between one or more core materials and one or more cladding materials of the light guide 408. In some examples, the core material(s) may include glass, silica, quartz, plastic, zirconium fluoride, chalcogenide, or combinations thereof. According to some examples, the cladding material(s) may include polyvinyl chloride (PVC), acrylic, polytetrafluoroethylene (PTFE), silicone or fluorocarbon rubber. The light guide 408 may, in some examples, include one or more optical fibers. As used herein, the terms “light guide” and “light pipe” may be used synonymously.

The apparatus 400 may additionally include a transparent backing layer 410. The transparent backing layer 410 may comprise glass (e.g., beads) and transparent epoxy in one illustrative configuration. Different weight percentage of glass and epoxy may impact the acoustic impedance and attenuation. Both acoustic impedance and attenuation may be tuned using the transparent backing layer 410 to affect overall optical transmission. The transparent backing layer 410, as with any other components included in the stack) may be constructed from materials that include: lithium niobate, lead magnesium niobate-lead titanate (PMN-PT), polyvinylidene fluoride tetrafluoroethylene (PVDF), and/or a copolymer film with an indium tin oxide coating.

A transparent piezo layer 412 may be positioned above the transparent backing layer 410. A patterned electrode layer 414 may be positioned on the transparent piezo layer 412. The implementation may allow light coming out of the VCSEL 406 to directly hit the tissue region (e.g., radial artery) 418 and a photoacoustic signal is generated. The generated photoacoustic wave travels isotopically and is received by the transparent ultrasonic receiver (Rx) board 402.

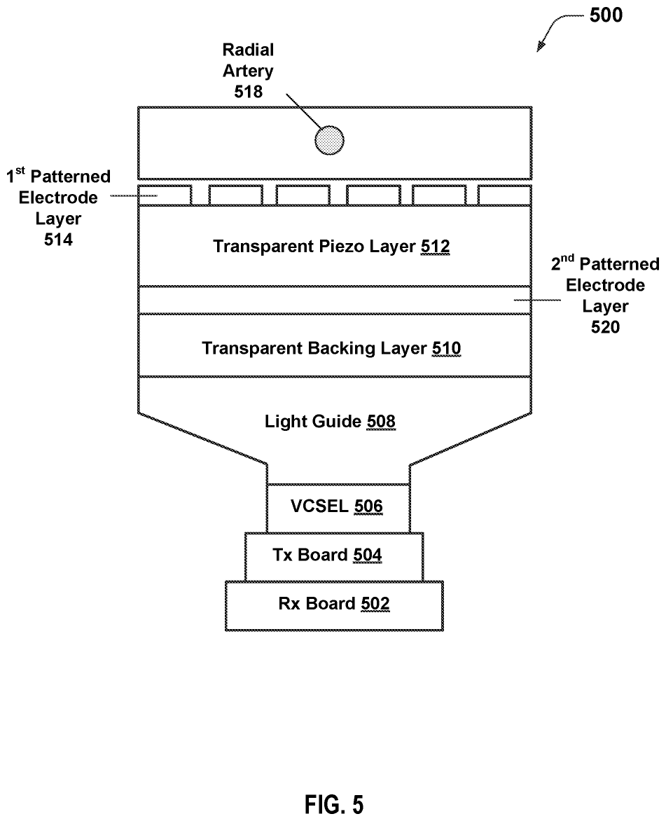

FIG. 5 is a block diagram of a side view of another photoacoustic sensor that differs from the apparatus 400 of FIG. 4 by including an additional patterned electrode layer. As with other disclosed examples, the types, numbers, and arrangements of elements shown in FIG. 5 are merely examples for illustrative purposes. Other examples may include different numbers of elements, types of elements, arrangements of elements, or combinations thereof.

Turning more particularly to the drawing, the apparatus 500 includes a receiver board 502 and a transmitter board 504. A VCSEL 506, which may generate pulsed laser transmissions, may be coupled to a light guide 508. The light guide 508 may include any suitable material, or combination of materials, for causing at least some of the light emitted by the VCSEL 506 to propagate within the light guide 508, for example due to total internal reflection between one or more core materials and one or more cladding materials of the light guide 508.

The apparatus 500 may additionally include a transparent backing layer 510. The transparent backing layer 510 may comprise glass (e.g., beads) and transparent epoxy in one illustrative configuration. Different weight percentage of glass and epoxy may impact the acoustic impedance and attenuation. Both acoustic impedance and attenuation may be tuned using the transparent backing layer 510 to affect overall optical transmission.

A transparent piezo layer 512 may be positioned above the transparent backing layer 510. A patterned electrode layer 514 may be positioned on the transparent piezo layer 512. A second patterned electrode layer 520 may be positioned on the transparent piezo layer 512 to provide for increased signal reception. The implementation may allow light coming out of the VCSEL 506 to directly hit the tissue region (e.g., radial artery) 518 and a photoacoustic signal is generated. The generated photoacoustic wave travels isotopically and is received by the transparent ultrasonic receiver (Rx) board 502.

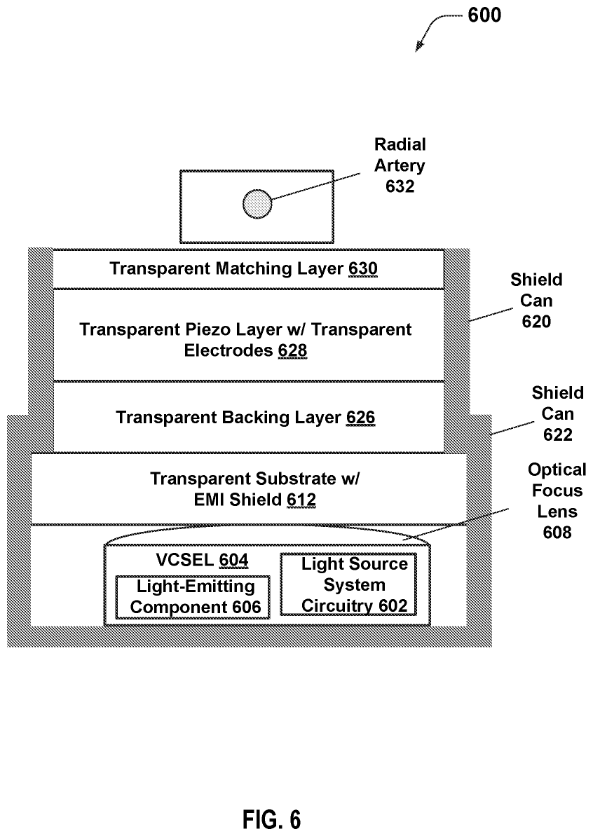

FIG. 6 shows some elements of a PAPG-capable sensor device having a receiver system stack that includes an optical focus lens positioned above a light source system. The implementation may allow co-alignment of optical and acoustic paths. As with other disclosed examples, the types, numbers, and arrangements of elements shown in FIG. 6 are merely examples for illustrative purposes. Other examples may include different numbers of elements, types of elements, arrangements of elements, or combinations thereof.

In this example, the apparatus 600 includes a light source system that comprising a VCSEL 604 having a light-emitting component 606 and light source system circuitry 602. The VCSEL 604 may generate pulsed laser transmissions. An optical focus lens 608 may be positioned above the VCSEL 604 to focus the laser pulse transmissions. According to this example, the apparatus 600 additionally includes a transparent substrate with an EMI shield 612. The EMI shield 612 may limit electromagnetic interference. The stack may be contained within shield cans 620, 622.

The apparatus 600 may additionally include a transparent backing layer 626. The transparent backing layer 626 may comprise glass and transparent epoxy in one illustrative configuration. The acoustic impedance and attenuation may be tuned using the transparent backing layer 626 to affect overall optical transmission.

A transparent piezo layer with transparent electrodes 628 may be positioned above the transparent backing layer 626. A transparent matching layer 630 may mitigate unwanted reflections of acoustic waves, thereby mitigating another type of noise. A radial artery 632 may be present proximate the transparent matching layer 630 to allow the system to determine blood pressure readings, among other data, from the detected acoustic waves.

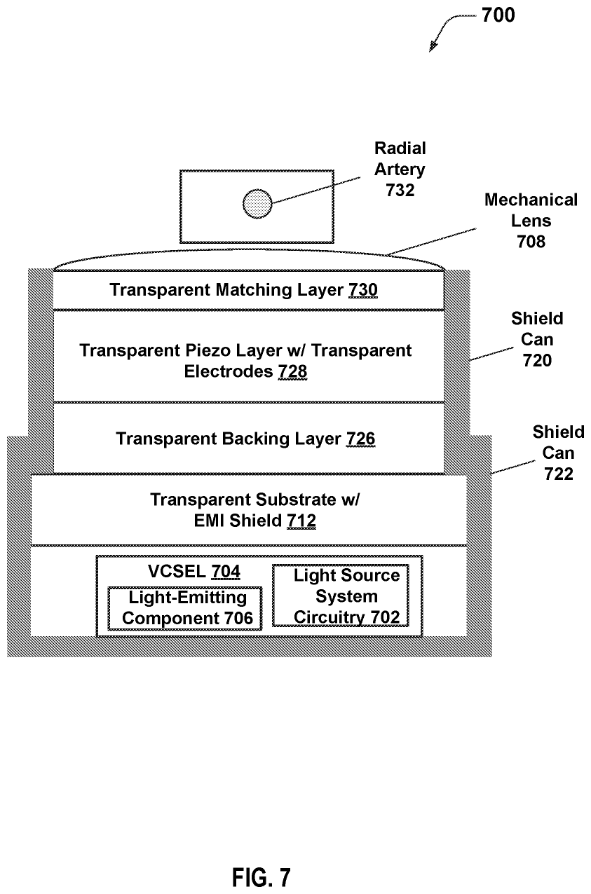

FIG. 7 shows some elements of a PAPG-capable sensor device having a receiver system stack that includes an optical focus lens positioned above the PAPG stack. The implementation may allow co-alignment of optical and acoustic paths. As with other disclosed examples, the types, numbers, and arrangements of elements shown in FIG. 7 are merely examples for illustrative purposes. Other examples may include different numbers of elements, types of elements, arrangements of elements, or combinations thereof.

In this example, the apparatus 700 includes a light source system that comprising a VCSEL 704 having a light-emitting component 706 and light source system circuitry 702. The VCSEL 704 may generate pulsed laser transmissions. According to this example, the apparatus 700 additionally includes a transparent substrate with an EMI shield 712. The EMI shield 712 may limit electromagnetic interference. The stack may be contained within shield cans 720, 722.

The apparatus 700 may additionally include a transparent backing layer 726. The transparent backing layer 726 may comprise glass and transparent epoxy in one illustrative configuration. The acoustic impedance and attenuation may be tuned using the transparent backing layer 726 to affect overall optical transmission.

A transparent piezo layer with transparent electrodes 728 may be positioned above the transparent backing layer 726. A transparent matching layer 730 may mitigate unwanted reflections of acoustic waves, thereby mitigating another type of noise. A mechanical lens 708 may be positioned above the transparent matching layer 730 to focus the laser pulse transmissions on a radial artery 732. The increased focus may facilitate health related readings from the from the detected acoustic waves.

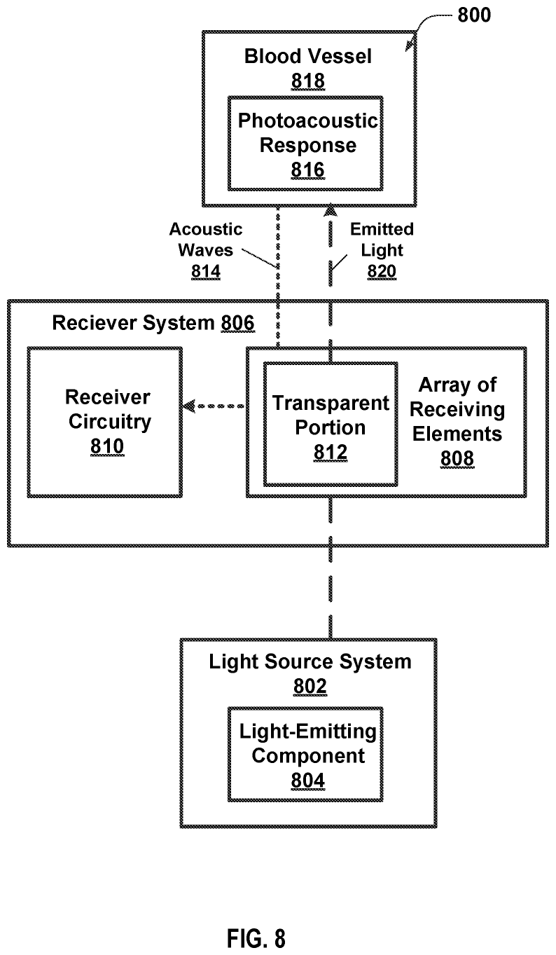

FIG. 8 is a block diagram of an apparatus 800 that includes a light source system 802. The light source system includes a light-emitting component 804. As shown in the illustration, a receiver system 806 includes an array of receiver elements 808 and receiver system circuitry 810. At least a portion 812 of the array of receiver elements 808 is transparent.

The receiver system 806 is configured to detect acoustic waves 814 corresponding to a photoacoustic response 816 of a blood vessel 818 to light 820 emitted by the light source system 802 through the transparent portion 812 of the array of receiver elements 808.

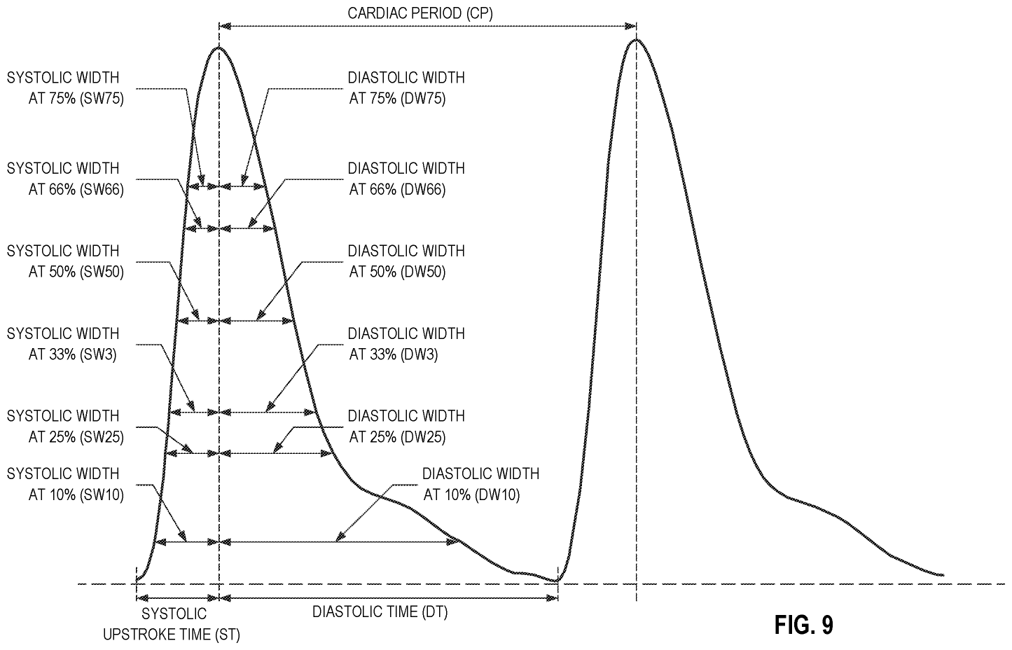

FIG. 9 shows examples of heart rate waveform (HRW) features that may be extracted according to some implementations of the systems and method described herein. The horizontal axis of FIG. 9 represents time, and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 9 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

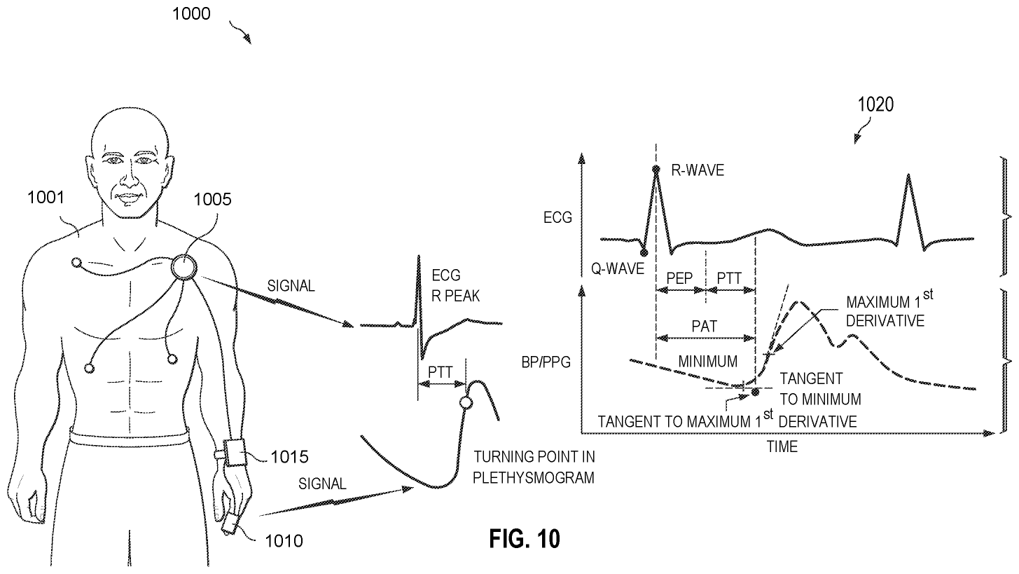

FIG. 10 shows examples of devices that may be used in a system for estimating blood pressure based, at least in part, on pulse transit time (PTT). As with other figures provided herein, the numbers, types and arrangements of elements are merely presented by way of example. According to this example, the system 1000 includes at least two sensors. In this example, the system 1000 includes at least an electrocardiogram sensor 1005 and a device 1010 that is configured to be mounted on a finger of the person 1001. In this example, the device 1010 is, or includes, an apparatus configured to perform at least some PAPG methods disclosed herein. For example, the device 1010 may be, or may include the apparatus 100 of FIG. 1 or a similar apparatus.

As noted in the graph 1020, the PAT includes two components, the pre-ejection period (PEP, the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 1020, in this example the beginning of a pulse arrival time (PAT) may be calculated according to an R-Wave peak measured by the electrocardiogram sensor 1005 and the end of the PAT may be detected via analysis of signals provided by the device 1010. In this example, the end of the PAT is assumed to correspond with an intersection between a tangent to a local minimum value detected by the device 1010 and a tangent to a maximum slope/first derivative of the sensor signals after the time of the minimum value.

There are many known algorithms for blood pressure estimation based on the PTT and/or the PAT, some of which are summarized in Table 1 and described in the corresponding text on pages 5-10 of Sharma, M., et al., Cuff-Less and Continuous Blood Pressure Monitoring: A Methodological Review (“Sharma”), in Multidisciplinary Digital Publishing Institute (MDPI) Technologies 2017, 5, 21, both of which are hereby incorporated by reference.

Some previously-disclosed methods have involved calculating blood pressure according to one or more of the equations shown in Table 1 of Sharma, or other known equations, based on a PTT and/or PAT measured by a sensor system that includes a PPG sensor. As noted above, some disclosed PAPG-based implementations are configured to distinguish artery HRWs from other HRWs. Such implementations may provide more accurate measurements of the PTT and/or PAT, relative to those measured by a PPG sensor. Therefore, disclosed PAPG-based implementations may provide more accurate blood pressure estimations, even when the blood pressure estimations are based on previously-known formulae.

Other implementations of the system 1000 may not include the electrocardiogram sensor 1005. In some such implementations, the device 1015, which is configured to be mounted on a wrist of the person 1001, may be, or may include, an apparatus configured to perform at least some PAPG methods disclosed herein. For example, the device 1015 may be, or may include the apparatus 200 of FIG. 2 or a similar apparatus. According to some such examples, the device 1015 may include a light source system and two or more ultrasonic receivers. One example is described below with reference to FIG. 15A. In some examples, the device 1015 may include an array of ultrasonic receivers.

In some implementations of the system 1000 that do not include the electrocardiogram sensor 1005, the device 1010 may include a light source system and two or more ultrasonic receivers. One example is described below with reference to FIG. 15B.

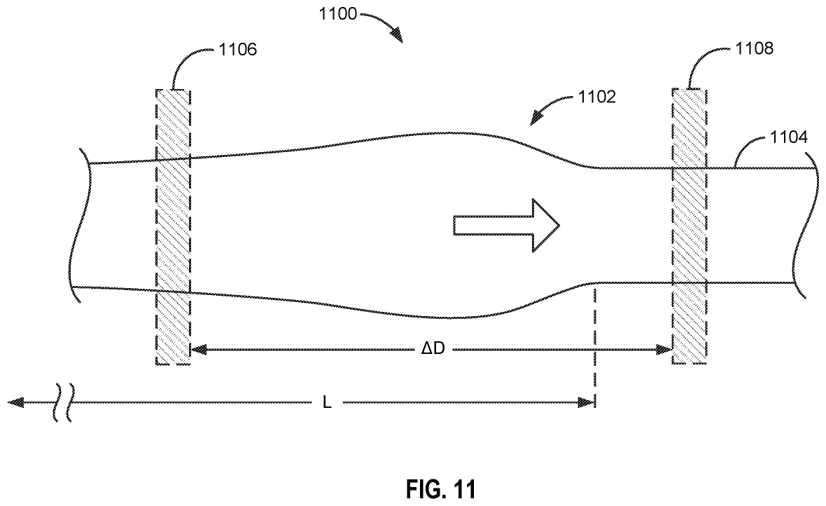

FIG. 11 shows a cross-sectional side view of a diagrammatic representation of a portion of an artery 1100 through which a pulse 1102 is propagating. The block arrow in FIG. 11 shows the direction of blood flow and pulse propagation. As diagrammatically shown, the propagating pulse 1102 causes strain in the arterial walls 1104, which is manifested in the form of an enlargement in the diameter (and consequently the cross-sectional area) of the arterial walls-referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. It will be appreciated that this PTT is different from the PTT that is described above with reference to FIG. 15. However, either version of the PTT may be used for the purpose of blood pressure estimation. Assuming that the physical distance AD between the first and the second physical locations is ascertainable, the PWV can be estimated as the quotient of the physical spatial distance AD traveled by the pulse divided by the time (PTT) the pulse takes in traversing the physical spatial distance AD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance AD through which the pulse propagates between the first physical location and the second physical location. More specifically, the PWV generally depends on a number of factors including the density of the blood p, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length AD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 1106 and a second arterial distension sensor 1108 proximate first and second physical locations, respectively, along an artery of interest. In some particular implementations, the first arterial distension sensor 1106 and the second arterial distension sensor 1108 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance AD of separation between the first arterial distension sensor 1106 and the second arterial distension sensor 1108 (and consequently the distance between the first and the second locations along the artery) can be in the range of about 1 centimeter (cm) to tens of centimeters-long enough to distinguish the arrival of the pulse at the first physical location from the arrival of the pulse at the second physical location, but close enough to provide sufficient assurance of arterial consistency. In some specific implementations, the distance AD between the first and the second arterial distension sensors 1106 and 1108 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance AD between the first and the second arterial distension sensors 1106 and 1108 can be less than or equal to 1 cm, for example, about 0.1 cm, about 0.25 cm, about 0.5 cm or about 0.75 cm. By way of reference, a typical PWV can be about 15 meters per second (m/s). Using an ambulatory monitoring device in which the first and the second arterial distension sensors 1106 and 1108 are separated by a distance of about 5 cm, and assuming a PWV of about 15 m/s implies a PTT of approximately 3.3 milliseconds (ms).

The value of the magnitude of the distance AD between the first and the second arterial distension sensors 1106 and 1108, respectively, can be preprogrammed into a memory within a monitoring device that incorporates the sensors (for example, such as a memory of, or a memory configured for communication with, the control system 106 that is described above with reference to FIG. 1). As will be appreciated by a person of ordinary skill in the art, the spatial length L of a pulse can be greater than the distance AD from the first arterial distension sensor 1106 to the second arterial distension sensor 1108 in such implementations. As such, although the diagrammatic pulse 1102 shown in FIG. 11 is shown as having a spatial length L comparable to the distance between the first arterial distension sensor 1106 and the second arterial distension sensor 1108, in actuality each pulse can typically have a spatial length L that is greater and even much greater than (for example, about an order of magnitude or more than) the distance AD between the first and the second arterial distension sensors 1106 and 1108.

### Sensing Architecture and Topology

In some implementations of the ambulatory monitoring devices disclosed herein, both the first arterial distension sensor 1106 and the second arterial distension sensor 1108 are sensors of the same sensor type. In some such implementations, the first arterial distension sensor 1106 and the second arterial distension sensor 1108 are identical sensors. In such implementations, each of the first arterial distension sensor 1106 and the second arterial distension sensor 1108 utilizes the same sensor technology with the same sensitivity to the arterial distension signal caused by the propagating pulses, and has the same time delays and sampling characteristics. In some implementations, each of the first arterial distension sensor 1106 and the second arterial distension sensor 1108 is configured for photoacoustic plethysmography (PAPG) sensing, e.g., as disclosed elsewhere herein. Some such implementations include a light source system and two or more ultrasonic receivers, which may be instances of the light source system 104 and the receiver system 102 of FIG. 1. In some implementations, each of the first arterial distension sensor 1106 and the second arterial distension sensor 1108 is configured for ultrasound sensing via the transmission of ultrasonic signals and the receipt of corresponding reflections. In some alternative implementations, each of the first arterial distension sensor 1106 and the second arterial distension sensor 1108 may be configured for impedance plethysmography (IPG) sensing, also referred to in biomedical contexts as bioimpedance sensing. In various implementations, whatever types of sensors are utilized, each of the first and the second arterial distension sensors 1106 and 1108 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph, or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude—and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 1106 and the second arterial distension sensor 1108, both the first arterial distension sensor 1106 and the second arterial distension sensor 1108 can be arranged, assembled, or otherwise included within a single housing of a single ambulatory monitoring device. As described above, the housing and other components of the monitoring device can be configured such that when the monitoring device is affixed or otherwise physically coupled to a subject, both the first arterial distension sensor 1106 and the second arterial distension sensor 1108 are in contact with or in close proximity to the skin of the user at first and second locations, respectively, separated by a distance AD, and in some implementations, along a stretch of the artery between which various arterial properties can be assumed to be relatively constant. In various implementations, the housing of the ambulatory monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the ambulatory monitoring device facilitates and enables long-term wearing and monitoring (for example, over days, weeks, or a month or more without interruption) of one or more biological characteristics of interest to obtain a better picture of such characteristics over extended durations of time, and generally, a better picture of the user's health.

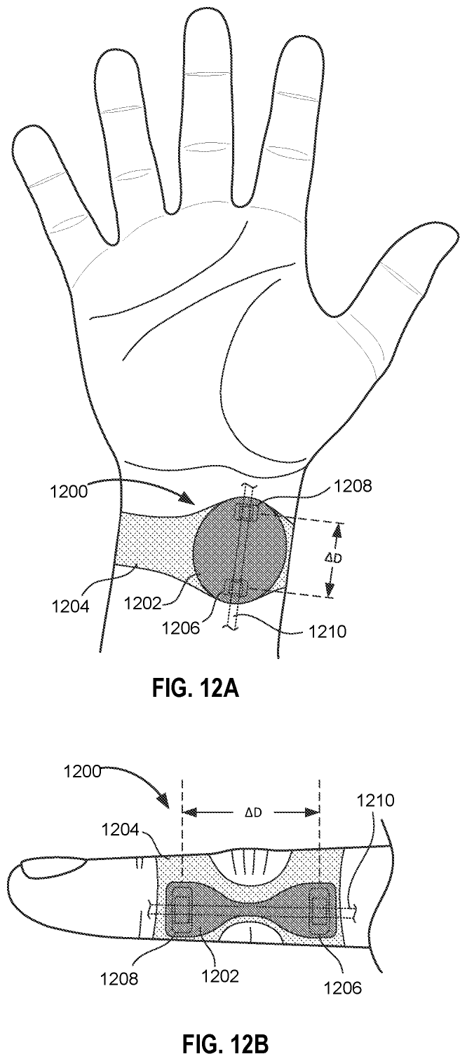

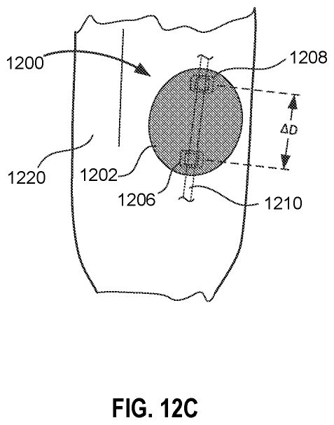

In some implementations, the ambulatory monitoring device can be positioned around a wrist of a user with a strap or band, similar to a watch or fitness/activity tracker. FIG. 12A shows an example ambulatory monitoring device 1200 designed to be worn around a wrist according to some implementations. In the illustrated example, the monitoring device 1200 includes a housing 1202 integrally formed with, coupled with, or otherwise integrated with a wristband 1204. The first and the second arterial distension sensors 1206 and 1208 may, in some instances, each include an instance of the ultrasonic receiver system 102 and a portion of the light source system 104 that are described above with reference to FIG. 1. In this example, the ambulatory monitoring device 1200 is coupled around the wrist such that the first and the second arterial distension sensors 1206 and 1208 within the housing 1202 are each positioned along a segment of the radial artery 1210 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 1206 and 1208 are separated by a fixed distance AD. In some other implementations, the ambulatory monitoring device 1200 can similarly be designed or adapted for positioning around a forearm, an upper arm, an ankle, a lower leg, an upper leg, or a finger (all of which are hereinafter referred to as “limbs”) using a strap or band.

FIG. 12B shows an example ambulatory monitoring device 1200 designed to be worn on a finger according to some implementations. The first and the second arterial distension sensors 1206 and 1208 may, in some instances, each include an instance of the ultrasonic receiver 102 and a portion of the light source system 104 that are described above with reference to FIG. 1.

In some other implementations, the ambulatory monitoring devices disclosed herein can be positioned on a region of interest of the user without the use of a strap or band. For example, the first and the second arterial distension sensors 1206 and 1208 and other components of the monitoring device can be enclosed in a housing that is secured to the skin of a region of interest of the user using an adhesive or other suitable attachment mechanism (an example of a “patch” monitoring device).

FIG. 12C shows an example ambulatory monitoring device 1200 designed to reside on an earbud according to some implementations. According to this example, the ambulatory monitoring device 1200 is coupled to the housing of an earbud 1220. The first and second arterial distension sensors 1206 and 1208 may, in some instances, each include an instance of the ultrasonic receiver 102 and a portion of the light source system 104 that are described above with reference to FIG. 1.

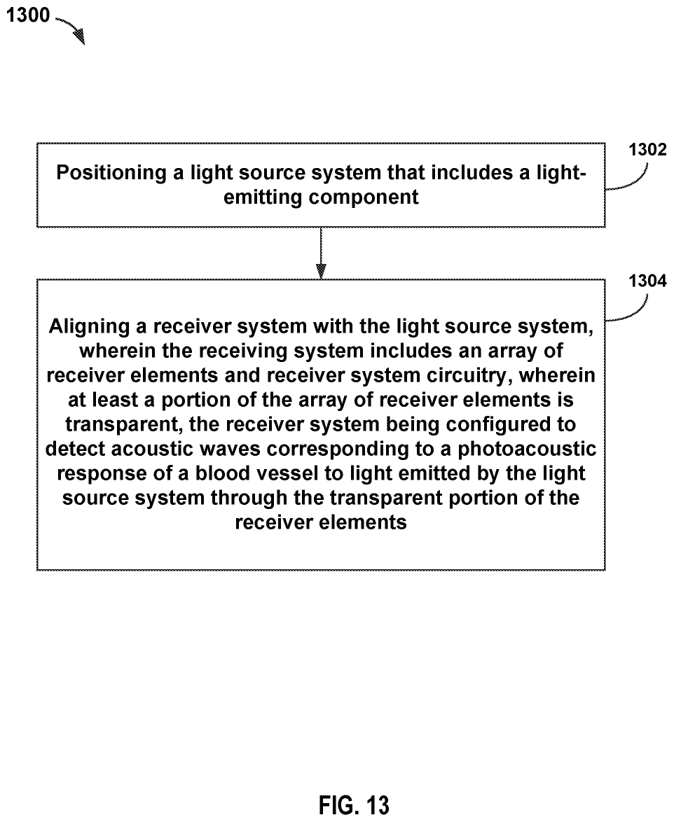

FIG. 13 is a flow diagram that shows examples of some disclosed operations. The blocks of FIG. 13 may, for example, be performed by the apparatus 100 of FIG. 1 or by a similar apparatus. For example, the blocks of FIG. 13 may be performed by the control system 106. As with other methods disclosed herein, the method outlined in FIG. 13 may include more or fewer blocks than indicated. Moreover, the blocks of methods disclosed herein are not necessarily performed in the order indicated. In some instances, one or more of the blocks shown in FIG. 13 may be performed concurrently.

Implementation examples are described in the following numbered clauses:
1. An apparatus, comprising:
a light source system including a light-emitting component; and
a receiver system including an array of receiver elements and receiver system circuitry, wherein at least a portion of the array of receiver elements is transparent, the receiver system being configured to detect acoustic waves corresponding to a photoacoustic response of a blood vessel to light emitted by the light source system through the transparent portion of the array of receiver elements.
2. The apparatus of clause 1, further comprising a platen having a transparent platen portion, wherein the platen is positioned in between the receiver system and where the blood vessel is presented.
3. The apparatus of clause 1 and 2, wherein a thickness of the platen ranges from 200 um to 400 um.
4. The apparatus of clauses 1-3, wherein the array of receiver elements comprises a transparent electrode layer.
5. The apparatus of clauses 1-4, wherein the receiver elements further comprise a piezoelectric layer having a first side on which the transparent electrode layer is included, and further comprising a patterned electrode layer included on a second opposite side of the piezoelectric layer.
6. The apparatus of clauses 1-5, further comprising a transparent matching layer positioned in between the receiver elements and the blood vessel.
7. The apparatus of clauses 1-6, further comprising a first transparent backing layer positioned in between the array of receiver elements and the light source system.
8. The apparatus of clauses 1-7, wherein the first transparent backing layer comprises at least one of glass or epoxy.
9. The apparatus of clauses 1-8, further comprising a second transparent backing layer proximate the first transparent backing layer.
10. The apparatus of clauses 1-9, wherein the first and second transparent backing layers have different material compositions.
11. The apparatus of clauses 1-10, wherein a first material composition of the second transparent backing layer is selected to affect at least one of an acoustic impedance or an attenuation based on a second material composition of the first transparent backing layer.
12. The apparatus of clauses 1-11, further comprising a transparent substrate with an electromagnetic interference (EMI) shield positioned in between the array of receiver elements and the light source system.
13. The apparatus of clauses 1-12, further comprising a light guide positioned in between the array of receiver elements and the light source system.
14. The apparatus of clauses 1-13, further comprising a lens positioned in between the array of receiver elements and the light source system.
15. The apparatus of clauses 1-14, further comprising a lens positioned in between the array of receiver elements and where the blood vessel is presented.
16. The apparatus of clauses 1-15, wherein the receiver system comprises at least one of: lithium niobate, lead magnesium niobate-lead titanate (PMN-PT), polyvinylidene fluoride tetrafluoroethylene (PVDF), or a copolymer film with an indium tin oxide coating.
17. An apparatus comprising:
a means for emitting a light;
a means for presenting an array of receiver elements, wherein at least a portion of the array of receiver elements is transparent; and
a means for detecting acoustic waves corresponding to a photoacoustic response of a blood vessel to the emitted light through the portion of the array of receiver elements that is transparent.
18. A method of manufacturing a photoacoustic sensor, the method comprising:
positioning a light source system including a light-emitting component; and
positioning a receiver system in proximity of the light source, wherein the light source system includes an array of receiver elements and receiver system circuitry, wherein at least a portion of the array of receiver elements is transparent, the receiver system being configured to detect acoustic waves corresponding to a photoacoustic response of a blood vessel to light emitted by the light source system through the transparent portion of the array of receiver elements.
19. The method of clause 18, further comprising positioning a platen in between the receiver system and where the blood vessel is presented, wherein the platen has a transparent platen portion.
20. The method of clauses 18 and 19, manufacturing the platen to have a thickness ranging from 200 um to 400 um.
21. The method of clauses 18-20, further comprising providing the array of receiver elements with a transparent electrode layer.
22. The method of clauses 18-21, further comprising including within the receiver elements a piezoelectric layer having a first side on which the transparent electrode layer is positioned and a second patterned electrode layer positioned on a second opposite side of the piezoelectric layer.
23. The method of clauses 18-22, further comprising positioning a transparent matching layer in between the receiver elements and the blood vessel.
24. The method of clauses 18-23, further comprising positioning a first transparent backing layer in between the array of receiver elements and the light source system.
25. The method of clauses 18-24, further comprising manufacturing the first transparent backing layer from glass and epoxy.
26. The method of clauses 18-25, further comprising positioning a second transparent backing layer proximate the first transparent backing layer.
27. The method of clauses 18-26, further comprising manufacturing the first and second transparent backing layers to have different material compositions.
28. The method of clauses 18-27, further comprising selecting a first material composition of the second transparent backing layer to affect at least one of an acoustic impedance or an attenuation based on a second material composition of the first transparent backing layer.
29. The method of clauses 18-28, further comprising positioning a transparent substrate with an electromagnetic interference (EMI) shield in between the array of receiver elements and the light source system.
30. The apparatus of clauses 18-29, further comprising positioning a light guide in between the array of receiver elements and the light source system.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

What is claimed is:

1. An apparatus, comprising:
a light source system including a light-emitting component; and
a receiver system including an array of receiver elements and receiver system circuitry, wherein at least a portion of the array of receiver elements is transparent, the receiver system being configured to detect acoustic waves corresponding to a photoacoustic response of a blood vessel to light emitted by the light source system through the transparent portion of the array of receiver elements.

2. The apparatus of claim 1, further comprising a platen having a transparent platen portion, wherein the platen is positioned in between the receiver system and where the blood vessel is presented.

3. The apparatus of claim 2, wherein a thickness of the platen ranges from 200 um to 400 um.

4. The apparatus of claim 1, wherein the array of receiver elements comprises a transparent electrode layer.

5. The apparatus of claim 4, wherein the receiver elements further comprise a piezoelectric layer having a first side on which the transparent electrode layer is included, and further comprising a patterned electrode layer included on a second opposite side of the piezoelectric layer.

6. The apparatus of claim 1, further comprising a transparent matching layer positioned in between the receiver elements and the blood vessel.

7. The apparatus of claim 1, further comprising a first transparent backing layer positioned in between the array of receiver elements and the light source system.

8. The apparatus of claim 7, wherein the first transparent backing layer comprises at least one of glass or epoxy.

9. The apparatus of claim 7, further comprising a second transparent backing layer proximate the first transparent backing layer.

10. The apparatus of claim 8, wherein the first and second transparent backing layers have different material compositions.

11. The apparatus of claim 8, wherein a first material composition of the second transparent backing layer is selected to affect at least one of an acoustic impedance or an attenuation based on a second material composition of the first transparent backing layer.

12. The apparatus of claim 1, further comprising a transparent substrate with an electromagnetic interference (EMI) shield positioned in between the array of receiver elements and the light source system.

13. The apparatus of claim 1, further comprising a light guide positioned in between the array of receiver elements and the light source system.

14. The apparatus of claim 1, further comprising a lens positioned in between the array of receiver elements and the light source system.

15. The apparatus of claim 1, further comprising a lens positioned in between the array of receiver elements and where the blood vessel is presented.

16. The apparatus of claim 1, wherein the receiver system comprises at least one of: lithium niobate, lead magnesium niobate-lead titanate (PMN-PT), polyvinylidene fluoride tetrafluoroethylene (PVDF), or a copolymer film with an indium tin oxide coating.

17. An apparatus comprising:
a means for emitting a light;
a means for presenting an array of receiver elements, wherein at least a portion of the array of receiver elements is transparent; and
a means for detecting acoustic waves corresponding to a photoacoustic response of a blood vessel to the emitted light through the portion of the array of receiver elements that is transparent.

18. A method of manufacturing a photoacoustic sensor, the method comprising:
positioning a light source system including a light-emitting component; and
positioning a receiver system in proximity of the light source, wherein the light source system includes an array of receiver elements and receiver system circuitry, wherein at least a portion of the array of receiver elements is transparent, the receiver system being configured to detect acoustic waves corresponding to a photoacoustic response of a blood vessel to light emitted by the light source system through the transparent portion of the array of receiver elements.

19. The method of claim 18, further comprising positioning a platen in between the receiver system and where the blood vessel is presented, wherein the platen has a transparent platen portion.

20. The method of claim 19, manufacturing the platen to have a thickness ranging from 200 um to 400 um.

21. The method of claim 18, further comprising providing the array of receiver elements with a transparent electrode layer.

22. The method of claim 21, further comprising including within the receiver elements a piezoelectric layer having a first side on which the transparent electrode layer is positioned and a second patterned electrode layer positioned on a second opposite side of the piezoelectric layer.

23. The method of claim 18, further comprising positioning a transparent matching layer in between the receiver elements and the blood vessel.

24. The method of claim 18, further comprising positioning a first transparent backing layer in between the array of receiver elements and the light source system.

25. The method of claim 24, further comprising manufacturing the first transparent backing layer from glass and epoxy.

26. The method of claim 24, further comprising positioning a second transparent backing layer proximate the first transparent backing layer.

27. The method of claim 26, further comprising manufacturing the first and second transparent backing layers to have different material compositions.

28. The method of claim 26, further comprising selecting a first material composition of the second transparent backing layer to affect at least one of an acoustic impedance or an attenuation based on a second material composition of the first transparent backing layer.

29. The method of claim 18, further comprising positioning a transparent substrate with an electromagnetic interference (EMI) shield in between the array of receiver elements and the light source system.

30. The apparatus of claim 18, further comprising positioning a light guide in between the array of receiver elements and the light source system.
